(19) United States
(12) Patent Application Publication
(10) Pub. No.: US 2010/0042149 A1
(43) Pub. Date: Feb. 18, 2010

(54) PELVIC OBLIQUITY CORRECTION INSTRUMENT

(76) Inventors: Nam T. CHAO, Marlborough, MA (US); Bill KENNEFICK, Plymouth, MA (US); Ross SYLVIA, Taunton, MA (US); Michael O'BRIEN, Pinecrest, FL (US); Randal BETZ, Ocean City, NJ (US); Peter NEWTON, San Diego, CA (US); Munish GUPTA, Carmichael, CA (US); Harry SHUFFLEBARGER, Coral Gables, FL (US); Suken SHAH, Greenville, DE (US); Praveen MUMMANENI, Hillsborough, CA (US); Per WESSBERG, Gothenberg (SE)

Correspondence Address: LAHIVE & COCKFIELD, LLP;FLOOR 30, SUITE 3000, ONE POST OFFICE SQUARE, BOSTON, MA, 02109, US

(21) Appl. No.: 12/193,694

(22) Filed: Aug. 18, 2008

(51) Int. Cl. A61B 17/70; A61F 5/00; A61B 17/56

(52) U.S. Cl. 606/246; 606/86 A; 606/60

(57) ABSTRACT

A pelvic obliquity correction instrument and methods of use are provided. The pelvic obliquity correction instrument includes a first engagement arm for engaging a portion of a spinal fixation system where the first engagement arm is adjustably coupled to a handle assembly. The pelvic obliquity correction instrument also includes a second engagement arm for engaging a second portion of the spinal fixation system. A surgeon exerts force on a first grip at a first end of the handle assembly and a second grip at a second end of the handle assembly to reposition a patient's pelvic bone. The pelvic obliquity correction instrument may provide a surgeon with greater precision, control and torque when rotating a patient's pelvic bone with respect to a superior portion of the patient's spine.

### FIELD OF INTEREST

The present invention relates to orthopedic surgical instruments and methods of use. More particularly, the present invention relates to an instrument for exerting force on a portion of a spinal fixation system attached to a patient's sacral pelvic construct.

### BACKGROUND

Spinal deformities, especially in patients with neuromuscular disease, often have a pelvic obliquity that makes ambulation difficult and prevents the patient from sitting comfortably in a wheelchair. Correction of this pelvic obliquity may require linking the pelvis to the spine using a rigid construct, often by means of a Luque-Galveston technique or an illiac screw pelvic fixation technique. These techniques require the pelvis to be rotated relative to the spine during the procedure of fitting the pelvic construct to the patient's sacral pelvic construct. Conventionally, the rotation is accomplished by exerting a force outside the surgical drapes and across the patient's hips and/or by applying contralateral distraction forces to the patient's shoulder and leg. Because the rotational forces are exerted on the soft tissue of the patient and not directly on the pelvis or the spine, the surgeon's precision and control of the rotation is limited. The stiffness of the deformity also makes it difficult for a surgeon to apply sufficient force directly to the bone of the pelvis and the spine to adjust a rotation while implants for the rigid construct are being inserted and tightened.

There is a need for an instrument for use in pelvic obliquity correction procedures that allows a surgeon to exert larger and more finely controlled forces on a pelvis and a superior portion of the spine. There is also a need for an instrument that results in more precise rotation and positioning of the pelvis with respect to the superior portion of the spine.

### SUMMARY

In accordance with one first aspect, a pelvic obliquity correction instrument is provided for exerting torque on a pelvic bone. The pelvic obliquity correction instrument includes a first engagement arm having an engagement end for engaging a first section of a spinal fixation system. The first engagement arm includes an adjustable coupling disposed at a coupling end. The pelvic obliquity correction instrument also includes a second engagement arm having an engagement end for engaging a second section of a spinal fixation system. The second engagement arm includes an adjustable coupling disposed at a coupling end. The pelvic obliquity correction instrument further includes a handle assembly with a first grip disposed at a first end and a second grip disposed at a second end. The handle assembly couples with the first engagement arm and the second engagement arm. The adjustable coupling of the first engagement arm adjustably couples with the handle assembly and is configured to adjust a distance between the first engagement arm and the second engagement arm.

In some embodiments, the handle assembly may include a first portion of the handle assembly adjustably coupled with the adjustable coupling of the first engagement arm and a second portion of the handle assembly coupled with the coupling end of the second engagement arm. The first portion and the second portion may be coupled by a joint. The joint may rotatably couple the first portion and the second portion of the handle assembly. The joint may be a polyaxial joint to permit rotation about more than one rotational axis. The pelvic obliquity instrument may further include a handle locking element to lock an orientation of the first portion of the handle assembly with respect to the second portion of the handle assembly.

In some embodiments, the adjustable coupling of the first engagement arm may be slidably coupled with the handle assembly. The adjustable coupling between the first engagement arm and the handle assembly may include a frictional drag feature to resist sliding of the first engagement arm relative to the handle assembly. The pelvic obliquity correction instrument may include a first arm lock to lock a position of the first engagement arm with respect to the handle assembly. The adjustable coupling of the second engagement arm may be slidably coupled with the handle assembly. The pelvic obliquity correction device may include a second arm lock to lock a portion of the second engagement arm with respect to the handle assembly.

In some embodiments, the handle assembly may have a polygonal cross section to resist rotation of the first engagement arm about a longitudinal axis of the handle assembly. An engagement end of the first engagement arm may be configured to engage a bone anchor. The engagement end of the first engagement arm may be configured to engage a rod of a spinal fixation system. The engagement end of the first engagement arm may be configured to engage a bar of a spinal fixation system. The engagement end of the first engagement arm may be configured to engage a plate. A slot may be formed in a side of the first engagement arm at the engagement end for engaging a spinal fixation element. A slot may be formed in the engagement end of the first engagement arm for engaging a spinal fixation element that extends toward the coupling end of the first engagement arm. The first engagement arm and the second engagement arm may be curved forming an engagement end separation distance between the engagement end of the first engagement arm and the engagement end of the second engagement arm that is smaller than a coupling end separation distance between the coupling end of the first engagement arm and the coupling end of the second engagement arm

In some embodiments, the first grip and the second grip may be separable from the handle assembly for replacement with a different first grip and a different second grip. The first extension arm may include a coupling piece disposed at a coupling end of the first extension arm and an extension piece separable from the coupling piece. The coupling piece and the extension piece may be configured to separate from each other and rejoin with the extension piece rotated along its longitudinal axis with respect to the coupling piece.

In accordance with another aspect, a method of using a pelvic obliquity correction instrument is provided to correct a pelvic obliquity. A distance between a coupling of a first engagement arm and a coupling of a second engagement arm of the pelvic obliquity correction instrument is adjusted to approximately equal a distance between a first section of a spinal fixation system attached to a patient and a second section of the spinal fixation system. The first section of the spinal fixation system is engaged with the engagement end of the first engagement arm. The second section of the spinal fixation system is engaged with an engagement end of the second engagement arm. Forces are exerted on the first grip and the second grip to reposition the patient's pelvis with respect to a superior portion of the patient's spine. A position of the pelvis is fixed with respect to the superior portion of the patient's spine. The pelvic obliquity correction instrument is then disengaged from the spinal fixation system.

In accordance with yet another aspect, a method of controlling a patient's spine during pedicle subtraction osteotomy is provided. A pelvic obliquity correction instrument is provided. The pelvic obliquity correction instrument includes first and second engagement arms, and a handle assembly which couples a adjustable coupling of the first engagement arm and a coupling of the second engagement arm. A first section of a spinal fixation system is engaged with an engagement end of the first engagement arm. A second section of the spinal fixation system is engaged with an engagement end of the second engagement arm. Lordosis is induced on the patient's spine by rotating the handle assembly or by moving the handle assembly along an up/down axis.

### BRIEF DESCRIPTION OF THE FIGURES

These and other features and advantages of the devices and methods disclosed herein will be more fully understood by reference to the following detailed description in conjunction with the attached drawings in which like reference numerals refer to like elements through the different views. The drawings illustrate principles of the instruments and methods disclosed herein and, although not to scale, show relative dimensions.

FIG. 1A illustrates a perspective view of an exemplary embodiment of a pelvic obliquity correction instrument;

FIG. 1B illustrates a perspective view of the pelvic obliquity correction instrument depicted in FIG. 1A, engaging an exemplary spinal fixation system of a patient;

FIG. 1C illustrates a perspective view of another exemplary embodiment of a pelvic obliquity correction instrument, engaging the exemplary spinal fixation system depicted in FIG. 1B;

FIG. 1D illustrates a perspective view of the pelvic obliquity correction instruction depicted in FIG. 1A, engaging another exemplary spinal fixation system of a patient;

FIG. 1E illustrates a perspective view of the pelvic obliquity correction instruction depicted in FIG. 1A, engaging yet another exemplary spinal fixation system of a patient;

FIG. 2A illustrates a side view of a first extension arm of an exemplary pelvic obliquity correction instrument;

FIG. 2B illustrates an exploded side view of the first extension arm depicted in FIG. 2A;

FIG. 2C illustrates an expanded perspective view of an engagement end of the first extension arm depicted in FIG. 2A;

FIG. 2D illustrates an expanded side view of a coupling end of the first extension arm depicted in FIG. 2A;

FIG. 3A depicts a perspective view of an exemplary adjustable coupling of a first engagement arm;

FIG. 3B depicts a front cross-sectional view of the adjustable coupling depicted in FIG. 3A;

FIG. 3C depicts a side view of the adjustable coupling depicted in FIG. 3A;

FIG. 4A depicts an enlarged perspective view of part of the exemplary pelvic obliquity correction instrument depicted in FIG. 1A illustrating a second detachable arm portion, separated from a second arm receiving portion and an adjustable coupling, according to aspects of an exemplary embodiment;

FIG. 4B depicts a perspective view of the pelvic obliquity correction instrument illustrating a different connection orientation for the second detachable arm portion, and illustrating a detachable second grip separated from the second portion of the handle assembly, according to aspects of an exemplary embodiment;

FIG. 5A depicts a side view of an arm portion of an engagement arm with a side engagement slot, according to aspects of an exemplary embodiment;

FIG. 5B depicts a perspective view of the arm portion depicted in FIG. 5A;

FIG. 6A depicts a perspective view of a pelvic obliquity correction instrument with a curved first engagement arm and a curved second engagement arm, according to aspects of an exemplary embodiment;

FIG. 6B depicts a perspective view of a pelvic obliquity correction instrument with a curved first engagement arm configured to engage a bone anchor and a curved second engagement arm configured engage a bone anchor, according to aspects of an exemplary embodiment;

FIGS. 7A and 7B graphically depict perspective views of two exemplary embodiments of a handle assembly of a pelvic obliquity correction instrument;

FIG. 8A graphically depicts a planar view of an orientation of an exemplary pelvic obliquity correction instrument in use during a pelvic obliquity correction procedure;

FIG. 8B graphically depicts a planar view of an orientation of an exemplary pelvic obliquity correction instrument in use after a pelvic obliquity has been corrected;

FIG. 8C graphically depicts a side view of an orientation of an exemplary pelvic obliquity correction instrument in use during a pelvic obliquity correction procedure;

FIG. 9A is a flow diagram of an exemplary method for correcting pelvic obliquity in a patient with a previously attached spinal fixation system using an exemplary pelvic obliquity correction instrument;

FIG. 9B is a flow diagram of another exemplary method for correcting pelvic obliquity in a patient with a previously attached spinal fixation system using an exemplary pelvic obliquity correction instrument;

FIG. 10A graphically depicts a perspective view of an exemplary pelvic obliquity correction instrument for use during a pedicle subtraction osteotomy (PSO) procedure;

FIG. 10B is a flow diagram of an exemplary method for controlling a patient's spine during a PSO procedure using an exemplary pelvic obliquity correction instrument;

FIG. 11A graphically depicts a perspective view of two exemplary pelvic obliquity correction instruments linked by an exemplary rack;

FIG. 11B graphically depicts a side view of an exemplary rack which is connected to an engagement arm of a first pelvic obliquity correction instrument and an engagement arm of a second pelvic obliquity correction instrument;

FIG. 11C graphical depicts a side view of an exemplary rack which includes a guide to control or limit translation of one of a first pelvic obliquity correction instrument relative to a second pelvic obliquity correction instrument; and

FIG. 11D graphically depicts a perspective view of first and second exemplary pelvic obliquity correction instruments whose left sides are linked by a first rack and whose right sides are linked by a second rack.

### DETAILED DESCRIPTION

Exemplary embodiments will now be described to provide an overall understanding of the principles of the structure, function, and use of the instruments and methods disclosed herein. One or more examples of these embodiments are illustrated in the accompanying drawings. Those skilled in the art will understand that the instruments and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Exemplary embodiments described herein provide pelvic obliquity correction instruments and methods of use. Exemplary embodiments of a pelvic obliquity correction instrument include engagement arms that couple to two sections of a spinal fixation system which is attached to the pelvis of a patient and a superior portion of the spine of the patient. An exemplary pelvic obliquity correction instrument includes a first grip at a first end of a handle assembly and a second grip at a second end of the handle assembly. A surgeon can use two spatially-separated hand grips to control a position of the pelvic obliquity correction instrument and the pelvis. This allows the surgeon to have fine control over the pelvic position and to exert larger forces on the pelvic bone in a controlled manner during a pelvic obliquity correction procedure.

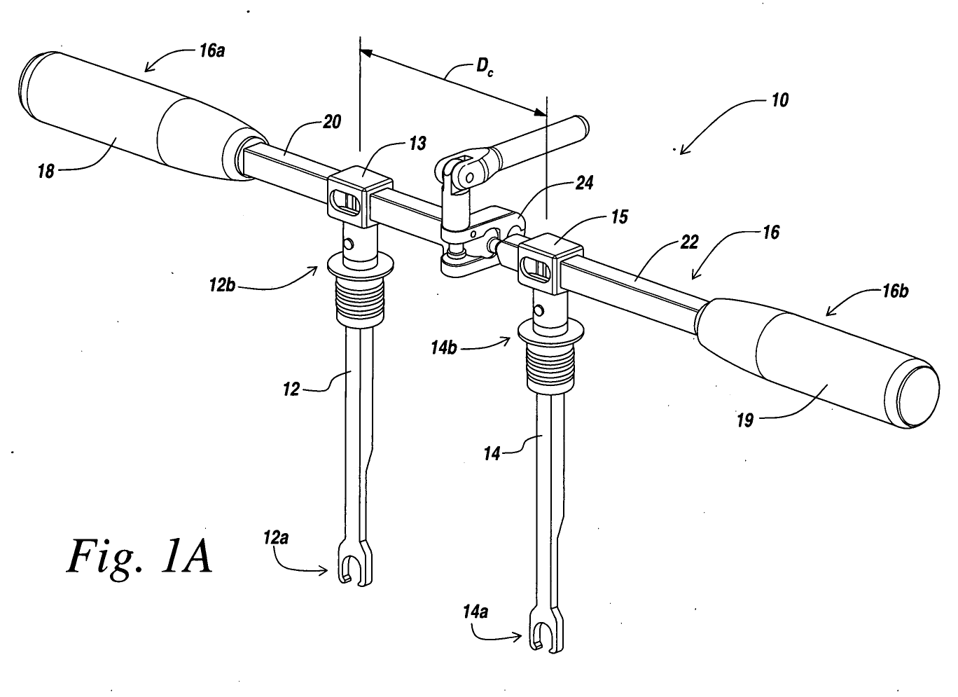

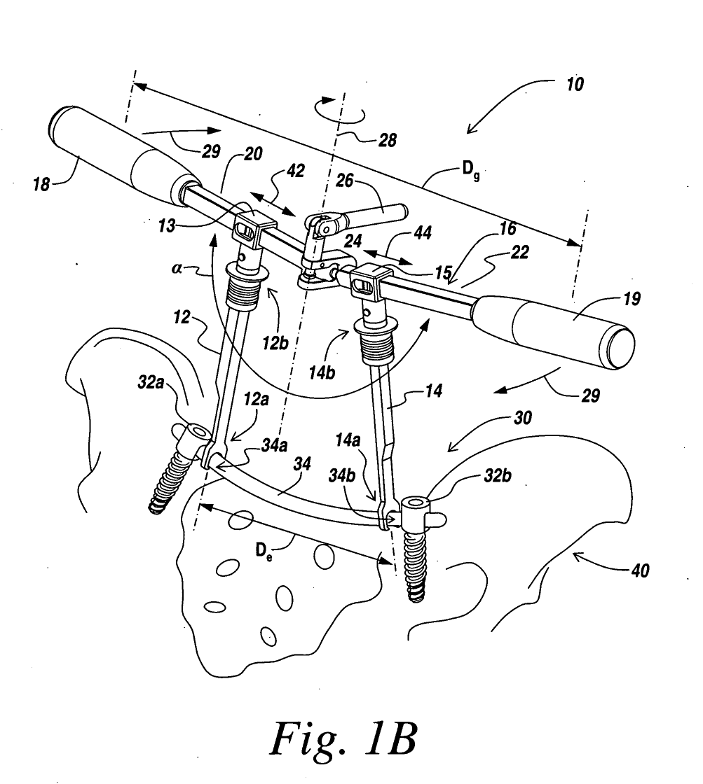

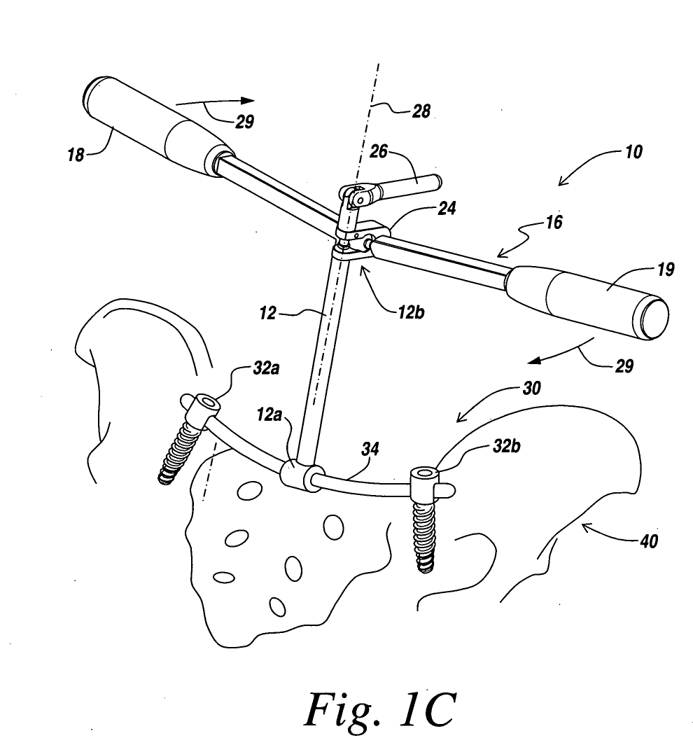

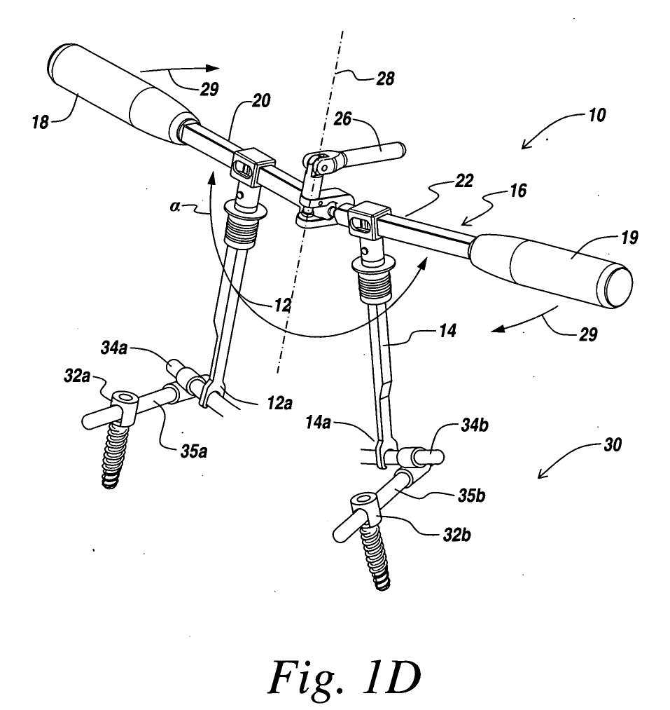

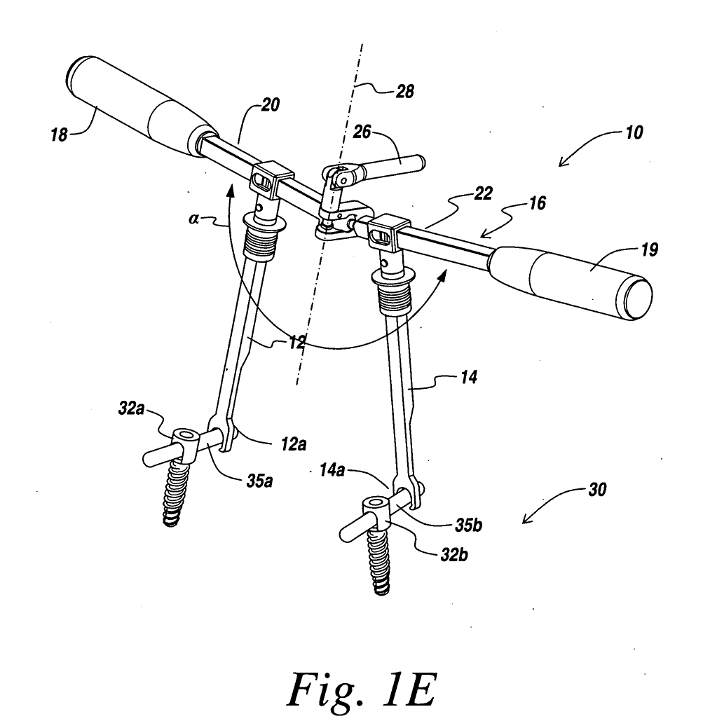

FIG. 1A illustrates a perspective view of an exemplary embodiment of a pelvic obliquity correction instrument 10 for positioning a patient's pelvis relative to the patient's superior portion of the spine. The pelvic obliquity correction instrument 10 includes a handle assembly 16, a first engagement arm 12 and a second engagement arm 14. The first and second engagement arms 12 and 14 may or may not be constructed from the same piece of metal as the handle assembly 16. The first engagement arm has an engagement end 12a for engaging a first section of a spinal fixation system 30. The first engagement arm 12 includes an adjustable coupling 13 disposed at a coupling end 12b. The second engagement arm 14 has an engagement end 14a for engaging a second section of a spinal fixation system 30. The second engagement arm 14 may also have an adjustable coupling 15 disposed at a coupling end 14b, in accordance with one aspect of an exemplary embodiment. The second engagement arm 14 may not have an adjustable coupling, in accordance with another aspect of an exemplary embodiment. The adjustable couplings 13 and 15 may be located at terminal ends of the first and second engagement arms 12 and 14, respectively. Alternatively, the adjustable couplings 13 and 15 may be located at non-terminal, intermediate points along the first and second engagement arms 12 and 14, respectively.

The handle assembly 16 has a first grip 18 disposed at a first end 16a of the handle assembly 16 and a second grip 19 disposed at a second end 16b of the handle assembly 16. The first and second grips 18 and 19 may be disposed at terminal ends of the handle assembly 16, as depicted in FIG. 1A. Alternatively, the first and second grips 18 and 19 may be disposed at non-terminal locations along the handle assembly 16.

In some embodiments, the adjustable coupling 13 of the first engagement arm 12 adjustably couples with the handle assembly 16 to adjust a distance D_{c} between the first engagement arm 12 and the second engagement arm 14. The adjustable coupling 15 of the second engagement arm 14 may also adjustably couple with the handle assembly 16 to adjust the distance D_{c} as depicted. The adjustable coupling 13 and/or the adjustable coupling 15 together with the handle assembly 16 may form an adjustable arm spacing mechanism for adjusting the spacing between the first engagement arm 12 and the second engagement arm 14.

In other embodiments, the distance D_{c} between the first and second engagement arms 12 and 14 is fixed. The first and second engagement arms 12 and 14 are not adjustable to vary the spacing between the arms. A pelvic obliquity correction instrument 10 designed according these embodiments may be provided in a kit containing several pelvic obliquity correction instruments, each with a different fixed distance D_{c}, i.e. with different arm-to-arm spacing.

According to aspects of an exemplary embodiment, the handle assembly 16 may include a first portion 20 adjustably coupled with the coupling end 12b of the first engagement arm 12 and a second portion 22 adjustably coupled with the coupling end 14b of the second engagement arm 14. The handle assembly 16 may also include a joint 24 coupling the first portion 20 with the second portion 22.

FIGS. 1B, 1D and 1E illustrate the exemplary pelvic obliquity correction instrument 10 in use. FIG. 1B depicts an exemplary embodiment of a spinal fixation system 30 including a first bone anchor 32a and a second bone anchor 32b which are both anchored in pelvic bone 40 and in a transverse rod 34. The pelvic obliquity correction instrument 10 may be adjusted to engage particular sections of the spinal fixation system 30. The distance between the first engagement arm 12 and the second engagement arm 14 may be adjusted by adjusting a position of the first engagement arm 12 relative to the handle assembly 16 as indicated by arrow 42 and/or by adjusting a position of the second engagement arm 14 with respect to the handle assembly 16 as indicated by arrow 44. As depicted, the adjustable coupling 13 of the first engagement arm 12 and adjustable coupling 15 of the second engagement arm 14 are adjusted by sliding the corresponding adjustable coupling (13 and 15 respectively) with respect to the handle assembly 16. A contact between the coupling ends 12b and 14b of the engagement arm and the handle assembly 16 may have sufficient friction to prevent sliding without externally applied force. Other suitable mechanisms for adjustably coupling the first engagement arm 12 and/or the second engagement arm 14 to the handle assembly 16 include, but are not limited to, a threaded mechanism, a rack and pinion, a ball plunger and detent, and a pawl and rachet. The adjustable coupling 13 allows the pelvic obliquity correction instrument 10 to adapt to different spacings between the first section 34a of the spinal fixation system 30 to be engaged and the second section 34b of the spinal fixation system 30 to be engaged.

Additionally, an angle a between the first portion 20 of the handle assembly 16 and the second portion 22 of the handle assembly 16 may be adjusted for different engagement geometries. According to aspects of an exemplary embodiment, the joint 24 that adjustably couples the first portion 20 of the handle assembly 16 with the second portion 22 of the handle assembly 16 may be a pivoting joint, a rotating joint, a polyaxial joint, such as the ball joint depicted, or another suitable joint coupling for adjusting the angle a. The joint 24 may include a joint lock 26 which allows a surgeon to lock the angle of the first portion 20 with respect to the second portion 22.

As depicted, the engagement end 12a of the first engagement arm 12 is engaging a first section 34a of the transverse rod 34, and the engagement end 14a of the second engagement arm 14 is engaging a second section 34b of the transverse rod 34. After the transverse rod 34 is engaged by both the first engagement arm 12 and the second engagement arm 14, the surgeon may lock the angle a of the first portion 20 of the handle assembly 16 with respect to the second portion 22 of the handle assembly 16 using the joint lock 26. The pelvic obliquity correction instrument 10 is rotated about axis 28 to exert torque on the spinal fixation system 30 and the pelvic bone 40 to which it is attached. Forces exerted by the surgeon on the first grip 18 and the second grip 19 are indicated by arrows 29. Because the distance D_{g} between the center of the first grip 18 and the center of the second grip 19 is greater than the distance Dₑ between the engagement end 12a of the first engagement arm 12 and the engagement end 14a of the second engagement arm 14, the handle assembly 16 acts as a lever to allow increased precision and control of torque applied to the spinal fixation system 30, which is attached to the pelvic bone 40. Forces on the first grip 18 and the second grip 19 may be exerted in reverse directions to exert torsion forces in an opposite direction.

FIG. 1C illustrates a perspective view of another exemplary embodiment of a pelvic obliquity correction instrument, engaging the exemplary spinal fixation system of FIG. 1B. The pelvic obliquity correction instrument 10 includes a single engagement arm 12. The engagement arm has an engagement end 12a for engaging substantially the middle of a transverse rod 34. The engagement arm has a coupling end 12b which rotatably couples to a joint 24 at the handle assembly 16. The pelvic obliquity correction instrument 10 is rotated about axis 28 to exert torque on the spinal fixation system 30 and the pelvic bone 40 to which it is attached.

Exemplary embodiments are not limited to the exemplary spinal fixation system 30 depicted in FIGS. 1B and 1C. For example, a surgeon may opt not to cross the sacro-iliac joint and will need to apply the rotation forces to sacral or pedicle screws at the end of the construct.

FIG. 1D depicts an exemplary embodiment of a spinal fixation system 30 which does not bilaterally link the pelvic construct through the bone anchors. The spinal fixation system 30 includes a first bone anchor 32a and a second bone anchor 32b which are both anchored in pelvic bone. The first bone anchor 32a is anchored to a first longitudinal rod 35a which is connected to a first transverse connector 34a. The second bone anchor 32b is anchored to a second longitudinal rod 35b which is connected to a second transverse connector 34b. The first and second transverse connectors 34a and 34b do not bilaterally link the pelvic construct. The engagement end 12a of the first engagement arm 12 engages the first transverse connector 34a, and the engagement end 14a of the second engagement arm 14 engages the second transverse connector 34b.

As depicted, the engagement end 12a of the first engagement arm 12 is engaging the first transverse connector 34a, and the engagement end 14a of the second engagement arm 14 is engaging the second transverse connector 34b. After the transverse connectors 34a and 34b are engaged by both the first engagement arm 12 and the second engagement arm 14, the surgeon may lock the angle a of the first portion 20 of the handle assembly 16 with respect to the second portion 22 of the handle assembly 16 using the joint lock 26. The pelvic obliquity correction instrument 10 is rotated about axis 28 to exert torque on the spinal fixation system 30 and the pelvic bone to which it is attached. Forces exerted by the surgeon on the first grip 18 and the second grip 19 are indicated by arrows 29. Forces on the first grip 18 and the second grip 19 may be exerted in reverse directions to exert torsion forces in an opposite direction.

FIG. 1E depicts yet another exemplary embodiment of a spinal fixation system 30 which does not include transverse rods and does not link the pelvic construct though the bone anchors. The spinal fixation system 30 includes a first bone anchor 32a and a second bone anchor 32b which are both anchored in pelvic bone. The first bone anchor 32a is anchored to a first longitudinal rod 35a, and the second bone anchor 32b is anchored to a second longitudinal rod 35b. The engagement end 12a of the first engagement arm 12 directly engages the first longitudinal rod 35a, and the engagement end 14a of the second engagement arm 14 directly engages the second longitudinal rod 35b.

As depicted, the engagement end 12a of the first engagement arm 12 is engaging the first longitudinal rod 35a, and the engagement end 14a of the second engagement arm 14 is engaging the second longitudinal rod 35b. After the first and second longitudinal rods 35a and 35b are engaged by both the first engagement arm 12 and the second engagement arm 14, the surgeon may lock the angle a of the first portion 20 of the handle assembly 16 with respect to the second portion 22 of the handle assembly 16 using the joint lock 26. The pelvic obliquity correction instrument 10 is rotated about axis 28 to exert torque on the spinal fixation system 30 and the pelvic bone to which it is attached. Forces exerted by the surgeon on the first grip 18 and the second grip 19 are indicated by arrows 29. Forces on the first grip 18 and the second grip 19 may be exerted in reverse directions to exert torsion forces in an opposite direction.

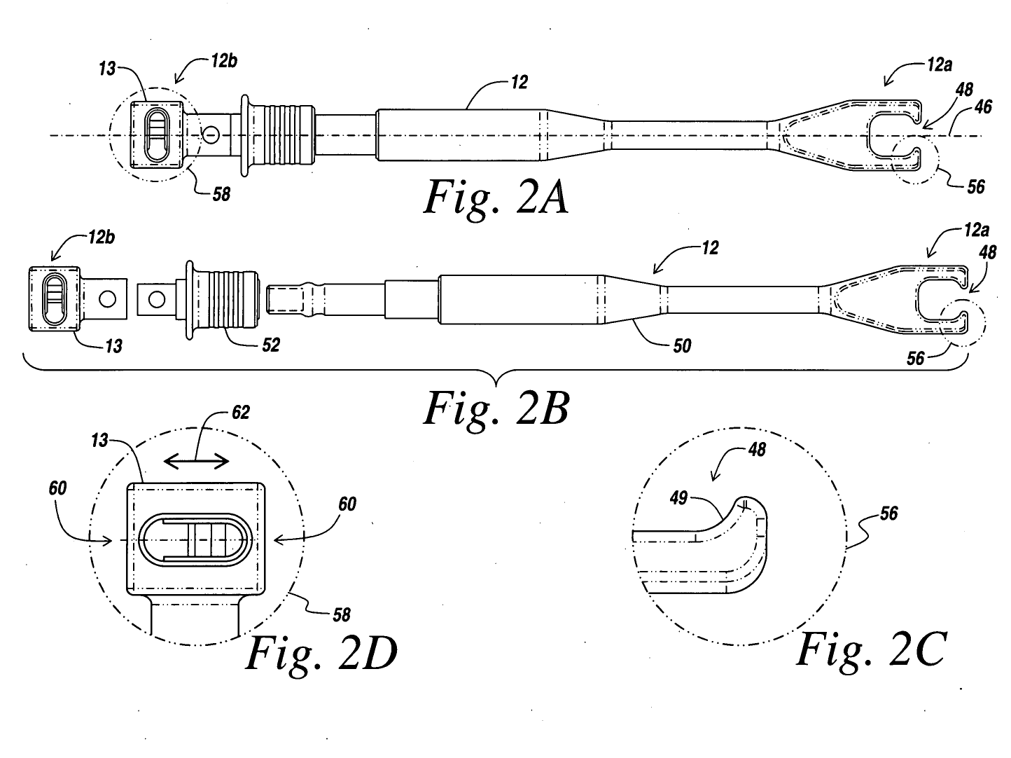

FIG. 2A illustrates a side view of the first engagement arm 12, which is suitable for engaging rods, bars, slots and other spinal fixation elements. In the exemplary embodiment depicted in FIGS. 1A, 1B, 1D and 1E, the first engagement arm 12 and the second engagement arm 14 have the same form. Thus, the descriptions of the first engagement arm 12 may also apply to the second engagement arm 14. The first engagement arm 12 has a central axis 46 that passes through a center of the first the engagement end 12a and a center of the coupling end 12b of the first engagement arm 12. According to an aspect of an exemplary embodiment, the engagement end 12a of the first engagement arm 12 has a slot 48 formed therein extending along the central axis 46 for engaging a section of a spinal fixation system.

FIG. 2B illustrates an exploded side view of the first engagement arm 12. According to aspects of an exemplary embodiment, the first engagement arm 12 may be formed of parts that include an arm portion 50, an arm receiving portion 52, and the adjustable coupling 13. The arm receiving portion 52 attaches to the adjustable coupling 13 and receives the arm portion 50. The arm receiving portion 52 may be configured to allow a surgeon to detach the arm portion 50 from the arm receiving portion 52 and reattach the arm portion 50 after rotating it with respect to the central axis 46. The arm receiving portion 52 may be configured to allow a surgeon to detach the arm portion 50 and attach an arm portion with a different structure. The arm receiving portion 52 and the arm portion 50 may be configured to engage with a “quick release” type of coupling.

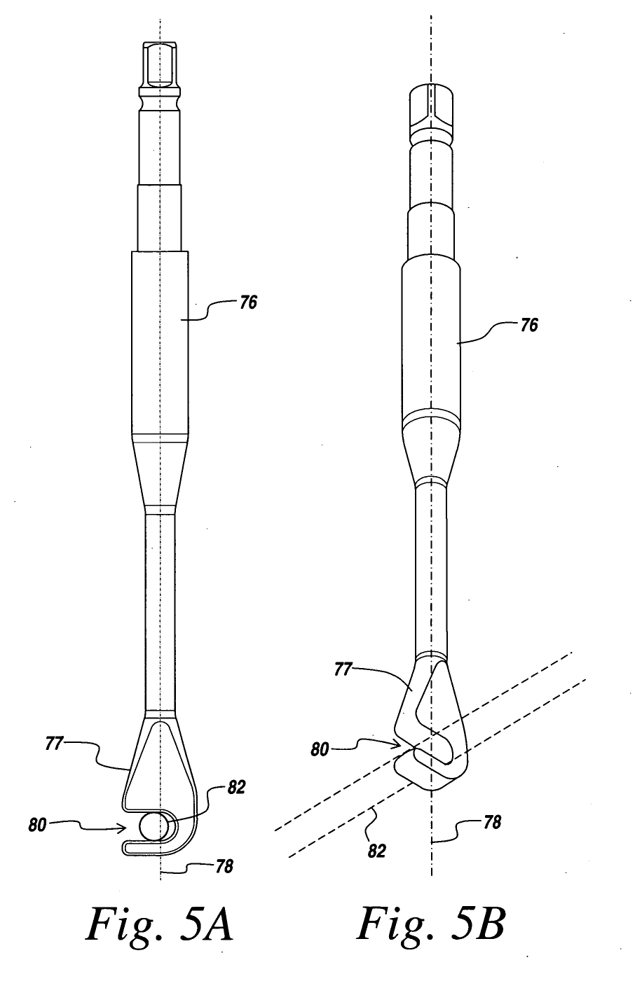

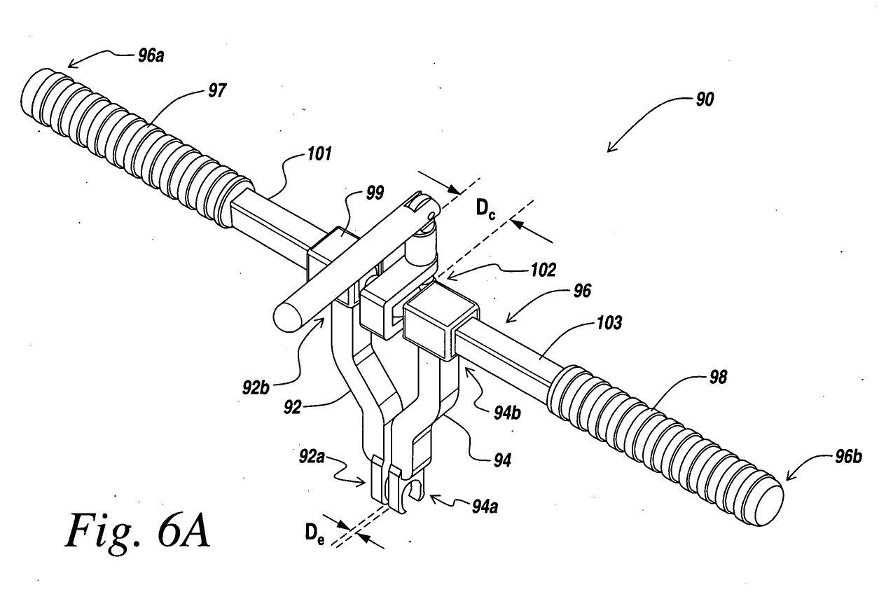

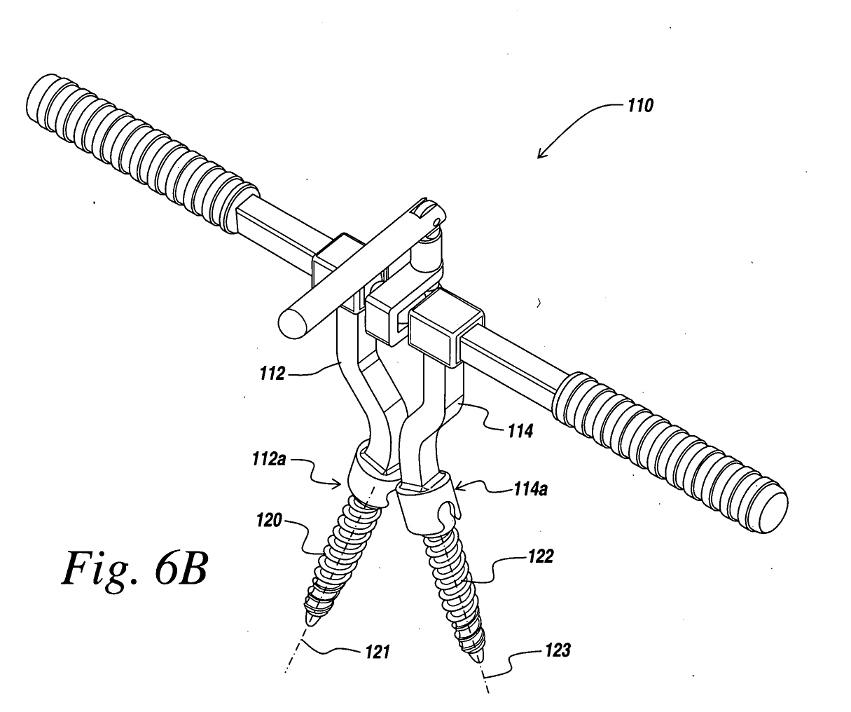

FIG. 2C depicts an enlarged perspective view 56 of the engagement end 12a. The slot 48 for engaging a section of a spinal fixation system 30 is undercut 49, as shown. The undercut 49 aids in preventing the first section 34a of the spinal fixation system 30 that is engaged by the first engagement arm 12 from sliding out of the slot 48 when the first engagement arm 12 is used to exert torque on the first section 34a of the spinal fixation system 30. The engagement end 12a may have different configurations for engaging various types of elements of a spinal fixation system 30, for engaging elements from various directions and for exerting torque about various axes. In an embodiment with a detachable arm portion 50, the detachable arm portion 50 may be exchanged for a different detachable arm portion having a different engagement end configuration. Other embodiments of the engagement end 12a are described with respect to FIGS. 5A, 5B, 6A and 6B below.

FIG. 2D depicts an enlarged side view 58 of the adjustable coupling 13 disposed at the coupling end 12b of the first engagement arm 12. The adjustable coupling 13 may have a coupling channel 60 for passing a portion of the handle assembly 16 therethrough. The coupling channel 60 may slidably couple with the handle assembly 16 allowing the first engagement arm 12 to slide with respect to the handle assembly as indicated by arrow 62.

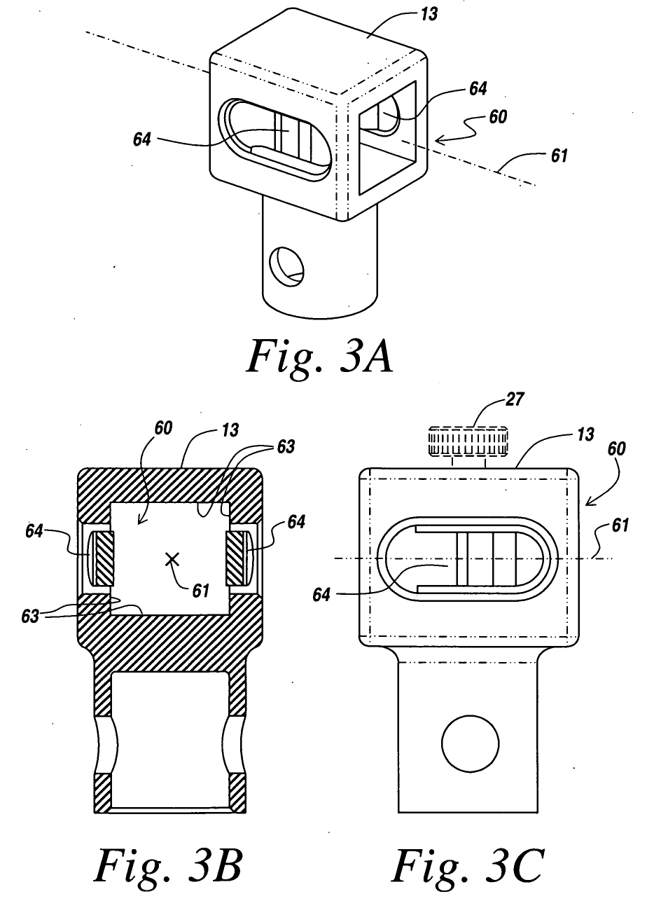

FIG. 3A illustrates a perspective view of the adjustable coupling 13 of the first engagement arm 12. A channel axis 61 extends along the coupling channel 60. FIG. 3B depicts a cross-sectional view of the adjustable coupling 13 along the channel axis 61. FIG. 3C depicts a side view of the adjustable coupling 13. According to aspects of an exemplary embodiment, the coupling channel 60 may have a cross-sectional shape with at least one flat side, such as a polygonal cross-section, for example, the square cross-section having four flat sides 63 as depicted in FIG. 3B. The flat sides 63 prevent the adjustable coupling 13 from rotating about the channel axis 61 with respect to the arm assembly. (See also FIG. 1A) According to aspects of an exemplary embodiment, the adjustable coupling 13 may include a frictional drag feature to resist sliding of the first engagement arm 12 relative to the handle assembly 16 in the absence of a force applied by the surgeon. The frictional drag feature may be in the form of curved tabs 64 which extend into the coupling channel 60 as depicted, in accordance with an aspect of an exemplary embodiment. The adjustable coupling 13 may optionally include a coupling lock 27 for locking a position of the first engagement arm 12 relative to the handle assembly 16, as depicted in FIG. 3C.

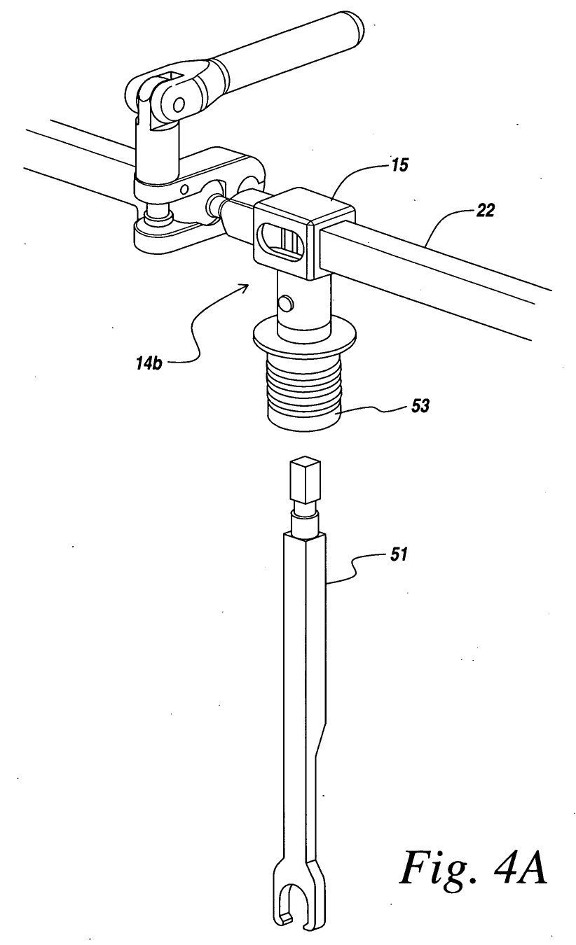

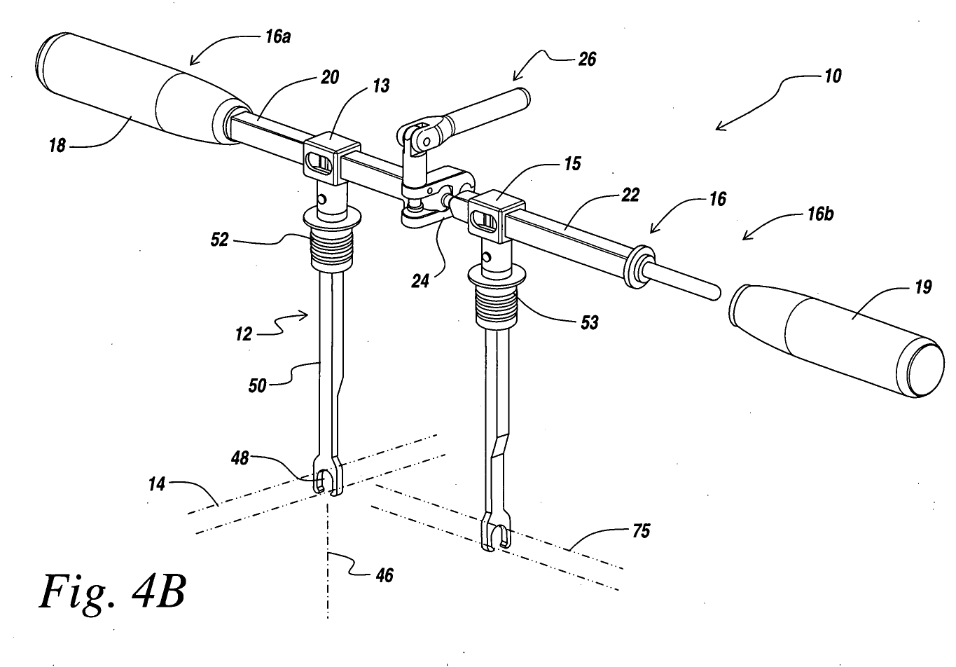

FIGS. 4A and 4B illustrate aspects of the configurability of the exemplary pelvic obliquity correction instrument 10. FIG. 4A is an enlarged perspective view of part of the exemplary pelvic obliquity correction instrument 10 illustrating a second detachable arm portion 51 separated from a second arm receiving portion 53 and the adjustable coupling 15 disposed at the coupling end 14b of the second engagement arm, according to aspects of an exemplary embodiment. The second arm receiving portion 53 and the second detachable arm portion 51 may be coupled with a “quick connect” type mounting as depicted, or may be coupled with any other coupling method known in the art that provides a secure connection between the second arm receiving portion 53 and the second detachable arm portion 51 and permits configurability by repositioning of and/or replacement of the second detachable arm portion 51.

As described above, a detachable first arm potion 50 may be detached from the arm receiving portion 52 and rotated about the central axis 46 of the engagement arm. FIG. 4B illustrates a perspective view of the pelvic obliquity correction instrument 10, with the first arm portion 50 coupled with the first arm receiving portion 52 in a configuration to engage a spinal fixation element 74 with a longitudinal orientation. The second arm portion 51 is coupled with the second arm receiving portion 53 in a different configuration to engage a spinal fixation element 75 with a transverse orientation. The slot 48 extending along the central axis 46 of the first engagement arm 12 allows the first engagement arm 12 to engage the spinal fixation element 74 from above. Likewise, the second engagement arm 14 is also configured for engagement from above the spinal fixation element 75.

As illustrated by FIG. 4B, the first grip 18 and the second grip 19 of the pelvic obliquity correction instrument 10 may be detachable for additional configurability, according to aspects of an exemplary embodiment. A surgeon may replace the first grip 18 and/or the second grip 19 with identical replacement grips or with replacement grips with different configurations. The first grip 18 and the second grip 19 may be detached before sterilization of the pelvic obliquity correction instrument 10.

As described above, the pelvic obliquity correction instrument may be configured and adapted by changing an orientation of the first portion 20 of the handle assembly 16 with respect to an orientation of the second portion 22 of the handle assembly 16. As depicted, the first portion 20 and the second portion 22 may be coupled by a polyaxial ball joint 24 that allows the first portion 20 to pivot relative to the second portion 22 in many different directions. The joint lock 26 allows a surgeon to lock an orientation of the first portion 20 with respect to the second portion 22 when the orientation is suitable for engaging the spinal fixation system 30.

FIGS. 5A and 5B illustrate an arm portion 76 of a different engagement arm configured for side engagement of a spinal fixation element 82. The arm portion 76 has a longitudinal axis 78 and a slot 80 formed on a side 77 of the arm portion 76 and extending perpendicular to the longitudinal axis 78. An exemplary pelvic obliquity correction instrument with two engagement arms of the same type in the same orientation, an exemplary pelvic obliquity correction instrument with two engagement arms of the same type in different orientations, and an exemplary pelvic obliquity instrument with two engagement arms of different types in different orientations all fall within the scope of the present invention.

A different exemplary embodiment of a pelvic obliquity correction instrument 90 is depicted in FIG. 6A. The pelvic obliquity correction instrument 90 has a first engagement arm 92 with an engagement end 92a and an adjustable coupling 99 disposed at a coupling end 92b. The pelvic obliquity correction instrument 90 also has a second engagement arm 94 with an engagement end 94a and a coupling end 94b for coupling with a handle assembly 96. The handle assembly 96 has a first grip 97 disposed at a first end 96a and a second grip 98 at a second end 96b. The adjustable coupling 99 adjustably couples the first engagement arm 92 with the handle assembly 96 to adjust a distance Dc between the engagement end 92a of the first engagement arm 92 and the engagement end 94a of the second engagement arm 94. In accordance with aspects of an exemplary embodiment, the first engagement arm 92 and/or the second engagement arm 94 may be curved as depicted. The curvature of the first engagement arm 92 and the second engagement arm 94 allows a distance Dₑ between the engagement end 12a of the first engagement arm 12 and the engagement end 94a of the second engagement arm 14 to be smaller than a distance D_{c} between the coupling ends. The distance Dc between the coupling ends cannot be reduced to zero due to the presence of a coupling joint 102 that joins a first portion 101 of the handle assembly 96 and a second portion 103 of the handle assembly 96. The curvature of the first engagement arm 92 and the second engagement arm 94 allow the distance Dₑ between the engagement ends to approach zero by sliding the first engagement arm 92 and the second engagement arm 94 with respect to the handle assembly 96.

Another exemplary embodiment of a pelvic obliquity correction instrument 110 is depicted in FIG. 6B. A first engagement arm 112 has an engagement end 112a configured to engage a first bone anchor 120. A second engagement arm 114 also has an engagement end 114a configured to engage a second bone anchor 122. A curvature of the first engagement arm 112 is configured to align the engagement end 112a of the first engagement arm 112 with a longitudinal axis 121 of the first bone anchor 120. Likewise, a curvature of the second engagement arm 114 is configured to align the engagement end 114a of the second engagement arm 114 with a longitudinal axis 123 of the second bone anchor 122.

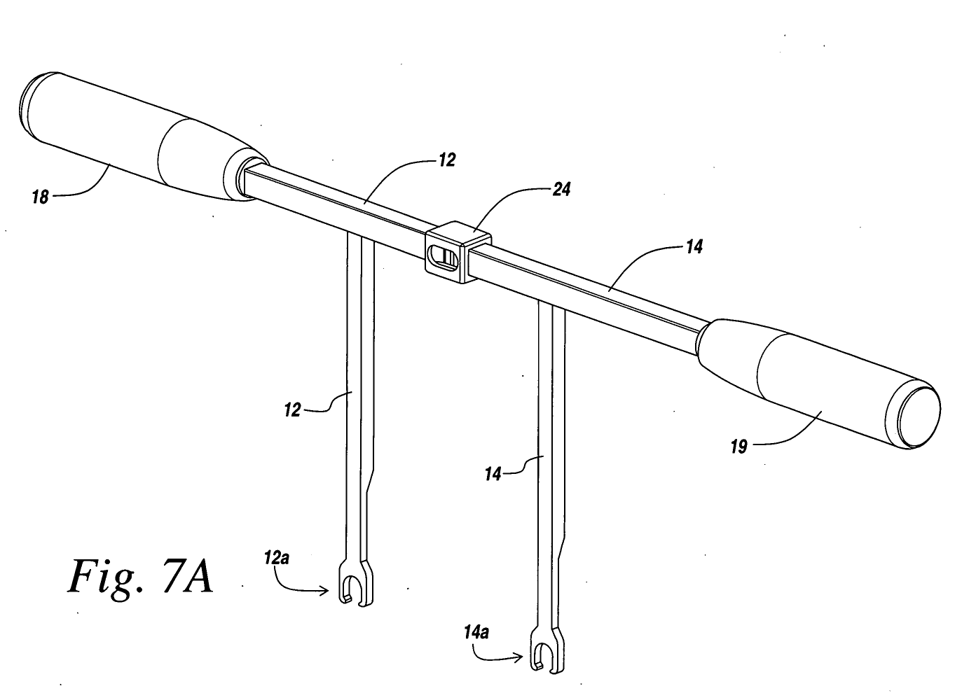

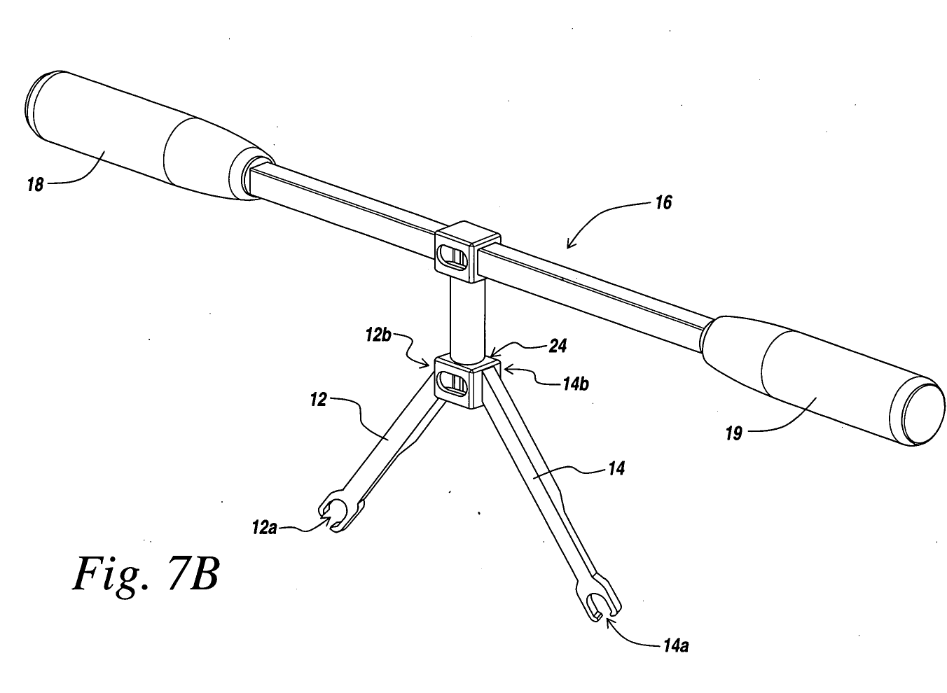

FIGS. 7A and 7B graphically depict perspective views of two exemplary embodiments of a handle assembly of a pelvic obliquity correction instrument 10 designed like a boat tiller.

In the exemplary embodiment depicted in FIG. 7A, the first and second engagement arms 12 and 14 of the handle assembly 16 are not adjustably coupled to two portions of the handle. Rather, the first engagement arm 12 is a single component including an arm and a handle portion. A first grip 18 is disposed at an end of the handle portion of the first engagement arm 12. The second engagement arm 14 is also a single component including an arm and a handle portion. A second grip 19 is disposed at an end of the handle portion of the second engagement arm 14. The first and second engagement arms, 12 and 14, are rotatable around a joint 24 to alter the spacing between the arms and/or an angle of the arms around a longitudinal axis.

In the exemplary embodiment depicted in FIG. 7B, rotatable coupling is provided between the first and second engagement arms 12 and 14, rather than linking the arms by a handle. A single handle 16 is connected to a rotatable joint 24. In one embodiment, a single grip is disposed in the middle of the handle 16. In another embodiment, a first grip 18 is disposed at a first end of the handle and a second grip 19 is disposed at a second end of the handle. The first engagement arm 12 is rotatably coupled to the second engagement arm 14 at the joint 24.

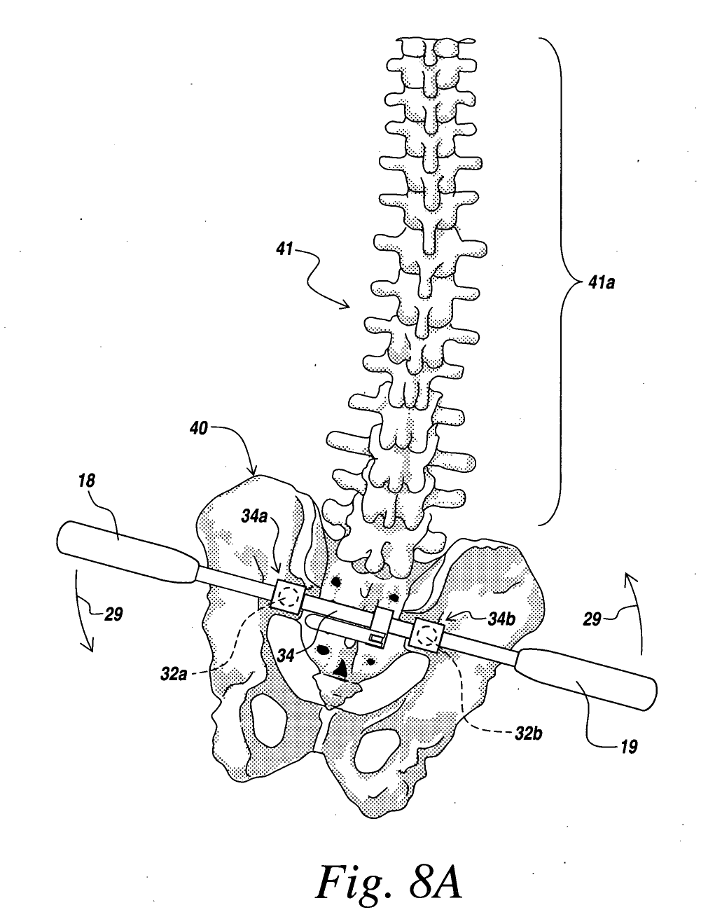

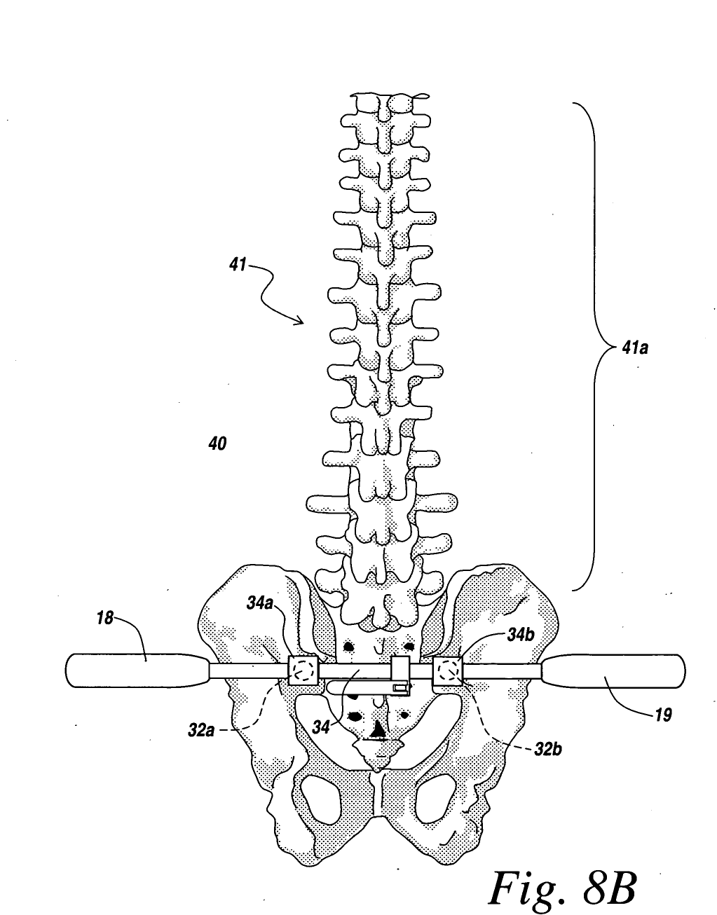

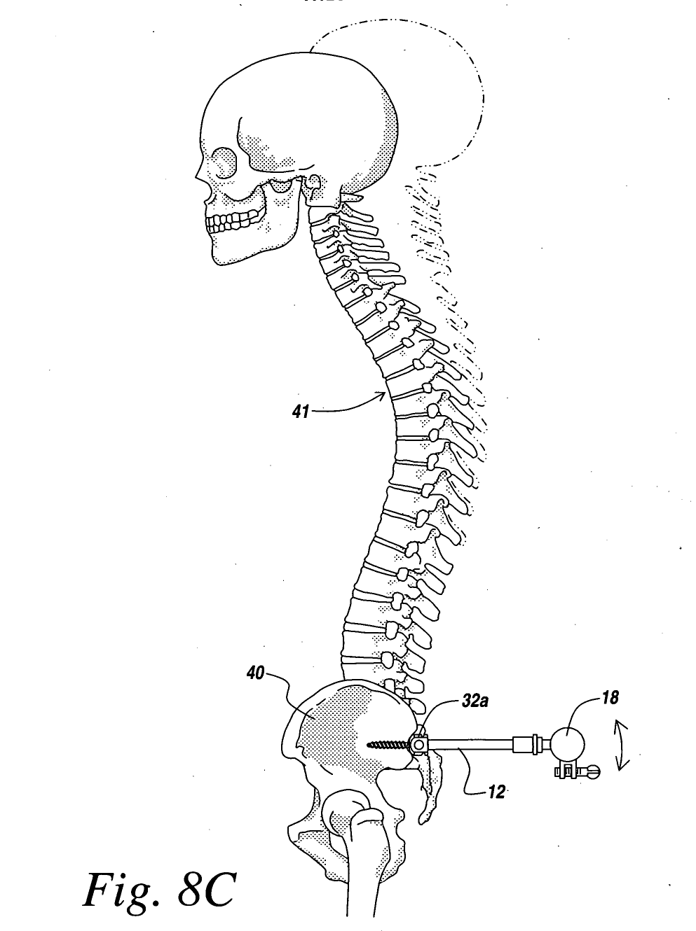

FIGS. 8A and 8B graphically depict planar views of an exemplary pelvic obliquity correction instrument 10 in use during a pelvic obliquity correction procedure. Solely for illustrative purposes, the procedure will be described with respect to the exemplary pelvic obliquity correction instrument 10 and the spinal fixation system 30 depicted in FIG. 1B and described above. FIG. 8A graphically depicts a planar view of a spine 41 and a pelvic bone 40 of a patient with a severe pelvic obliquity as indicated by the curvature of the patient's spine 41. A spinal fixation system 30 including a first bone anchor 32a, a second bone anchor 32b and a transverse rod 34 is attached to the patient. The pelvic obliquity correction instrument 10 is adapted to engage a first section 34a of the transverse rod 34 and a second section 34b of the transverse rod 34. After the transverse rod 34 is engaged, a surgeon uses the first grip 18 and a second grip 19 to exert forces on the pelvic bone 40 to straighten the spine 41. The forces 29 exerted on the first grip 18 and on the second grip 19 both translate and rotate the pelvic bone 40 with respect to a superior portion 41a of the spine until the spine 41 is straightened to the desired degree as depicted in FIG. 8B. The distance between the grips Dg allows the surgeon additional leverage when rotating the pelvic bone 40. Additionally, the distance between the grips D_{g} allows the surgeon to exercise greater control over the motion of the pelvic bone 40.

FIG. 8B graphically depicts a planar view of the spine 41 and the pelvic bone 40 of the patient after the pelvic obliquity has been corrected, according to aspects of an exemplary embodiment. FIG. 8C graphically depicts a side view of the spine 41 and the pelvic bone 40 during a pelvic obliquity correction procedure, according to aspects of an exemplary embodiment.

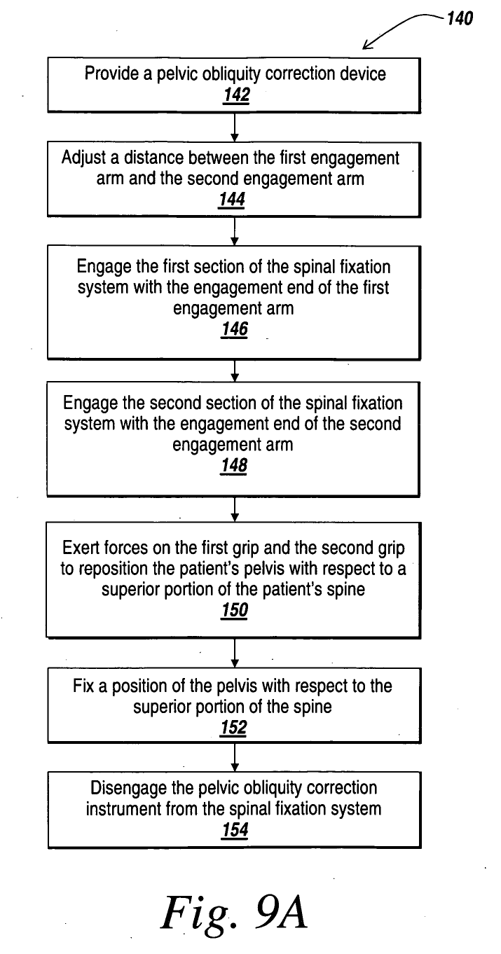

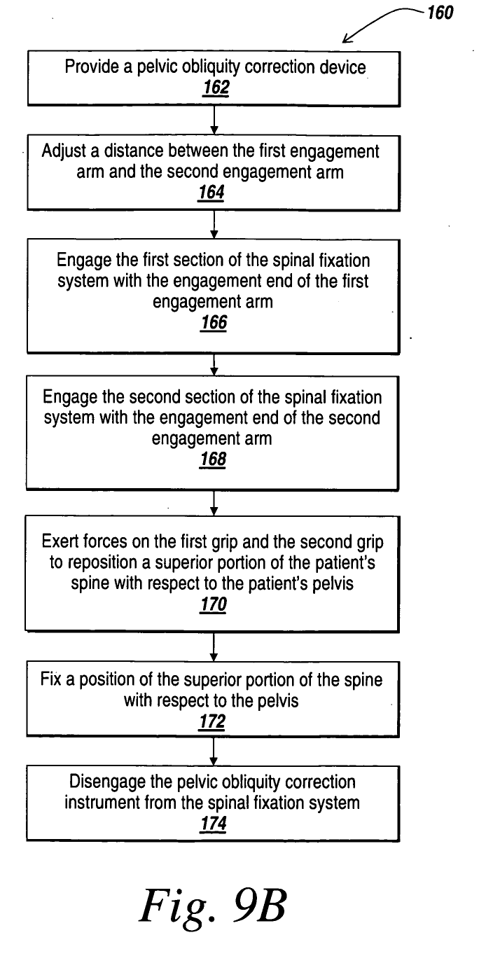

FIG. 9A is a flow diagram of an exemplary method 140 for correcting pelvic obliquity in a patient with a previously attached spinal fixation system 30 using a pelvic obliquity correction instrument 10. Solely for illustrative purposes, the method will be described with respect to the exemplary pelvic obliquity correction instrument 10 depicted in FIG. 1A and the spinal fixation system 30 depicted in FIGS. 1B, 8A and 8B. The pelvic obliquity correction instrument 10 is provided (step 142). Providing the pelvic obliquity correction instrument 10 may include selecting a suitable first arm portion 50 and attaching the selected first arm portion 50 to an arm receiving portion 52 of the first engagement arm 12 in a desired angular orientation. (See also FIG. 4A) A distance between the first engagement arm 12 and the second engagement arm 14 is adjusted to approximately equal a distance between a first section 34a of a spinal fixation system 30 and a second section 34b of a spinal fixation system 30 (step 144). The distance between the first engagement arm 12 and the second engagement arm 14 may be adjusted by sliding the first adjustable coupling 13 of the first engagement arm 12 with respect to the handle assembly 16 and/or by sliding the second adjustable coupling 15 of the second engagement arm 14. The distance between the first engagement arm 12 and the second engagement arm 14 may also be adjusted by rotating the first grip 18 and the second grip 19 around the joint 24.

The first section 34a of the spinal fixation system 30 is engaged with the engagement end 12a of the first engagement arm 12 (step 146). The second section 34b of the spinal fixation system 30 is engaged with the engagement end 14a of the second engagement arm 14 (step 148). After the spinal obliquity correction device is engaged with the first section 34a and the second section 34b of the spinal fixation system 30, the surgeon exerts forces on the first grip 18 and the second grip 19 to reposition the patient's pelvic bone 40 with respect to a superior portion 41a of the patients spine 41 (step 150). After the patient's pelvic bone 40 has been repositioned, the surgeon fixes a position of the pelvic bone 40 with respect to the superior portion 41a of the spine 41 (step 152). The surgeon finally disengages the pelvic obliquity correction instrument 10 from the spinal fixation system 30 (step 154).

FIG. 9B is a flow diagram of another exemplary method 160 for correcting pelvic obliquity in a patient with a previously attached spinal fixation system 30 using a pelvic obliquity correction instrument 10. Steps 162, 164, 166, 168 and 174 in method 160 are the same as steps 142, 144, 146, 148 and 154 in method 140 depicted in FIG. 9A. After the spinal obliquity correction device is engaged with the first section 34a and the second section 34b of the spinal fixation system 30, the surgeon exerts forces on the first grip 18 and the second grip 19 to reposition a superior portion 41a of the patient's spine 41 with respect to the patient's pelvic bone 40 (step 170). After a superior position 41a of the patient's spine 41 has been repositioned, the surgeon fixes a position of the superior position 41a with respect to the patient's pelvic bone 40 (step 172).

Although an exemplary method has been described in which first engagement arm 12 engages a portion of a transverse rod 34 and the second engagement arm 14 engages another portion of the transverse rod 34, in exemplary methods the first engagement arm 12 and the second engagement arm 14 may engage many different types of spinal fixation elements including but not limited to rods, plates, bars, bone screws bolts, etc. In some embodiments, the first engagement arm 12 may engage a different type of element than an element engaged by the second engagement arm 14. In other embodiments, the first engagement arm 12 may engage the same type of element as an element engaged by the second engagement arm 14.

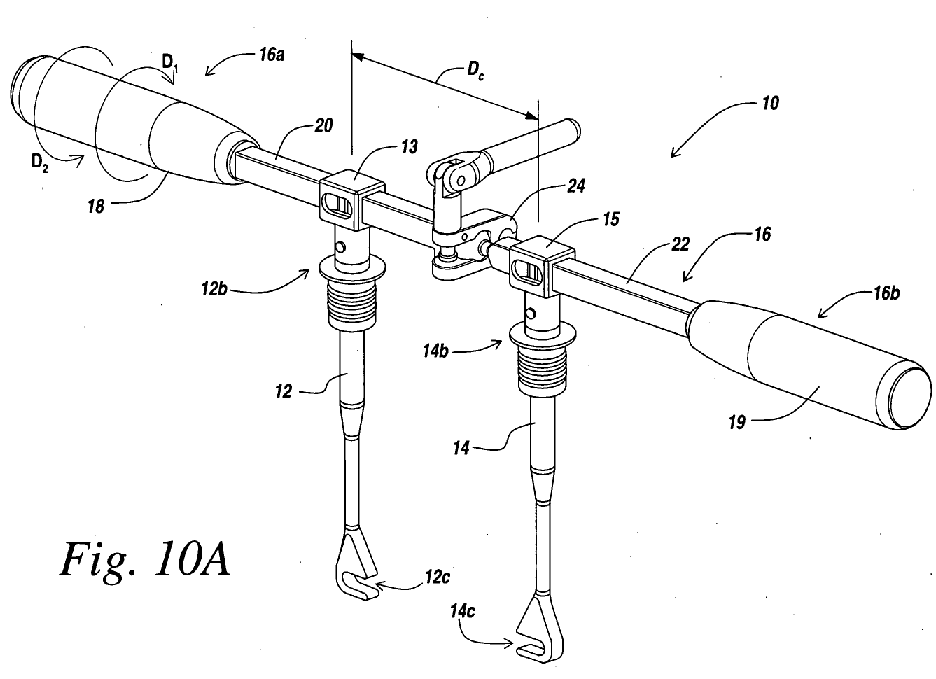

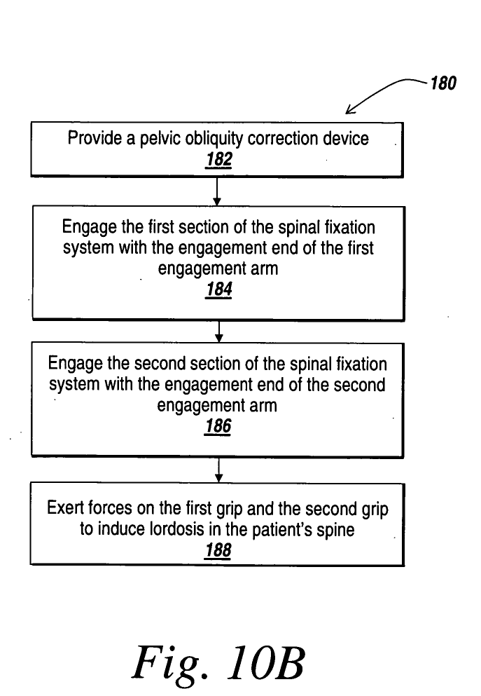

Exemplary embodiments of pelvic obliquity correction instrument 10 can also be used to control a patient's spine during pedicle subtraction osteotomy (PSO). Controlling a patient's spine during PSO is difficult and generally done manually by placing towels under the patient's thighs or manipulating the operating table to induce lordosis of the spine. A pelvic obliquity correction instrument 10, provided according to aspects of an exemplary embodiment, can be used to control the spine during a PSO procedure. FIG. 10A graphically depicts a perspective view of an exemplary pelvic obliquity correction instrument 10 for use during a PSO procedure.

This embodiment of the pelvic obliquity correction instrument 10 includes alternate engagement arms 12 and 14 that connect to the longitudinal rod below the PSO region. The first engagement arm 12 has a side loading slot 12c, and the second engagement arm 14 has a side loading slot 14c. In one embodiment, the side loading slots 12c and 14c face each other. The loading slots 12c and 14c engage the patient's spine. In one embodiment, the loading slots engage longitudinal rods below the PSO region. In another embodiment, the loading slots directly engage screw heads on the patient's spine.

FIG. 10B is a flow diagram of an exemplary method 180 for controlling a patient's spine during a PSO procedure using an exemplary pelvic obliquity correction instrument 10. Solely for illustrative purposes, the method will be described with respect to the exemplary pelvic obliquity correction instrument 10 depicted in FIG. 10A. The pelvic obliquity correction instrument 10 is provided (step 182). Providing the pelvic obliquity correction instrument 10 may include engaging a first section of a spinal fixation system attached to the patient's spine with the loading slot 12c of the first engagement arm 12 (step 184), and engaging a second section of the spinal fixation system with the loading slot 14c of the second engagement arm 14 (step 186). After the spinal obliquity correction device is engaged with the spinal fixation system, the handle assembly 16 is pushed up/down and/or rotated in a direction D₁ or D₂ to induce lordosis in the spine (step 188). Forces can be applied to the grips on the handle assembly to achieve either the up/down or rotation motions of the handle assembly. Optionally, instruments may be linked above and below the PSO region to adjust angulation of the first and second engagement arms 12 and 14 for precise mechanical control of the spine during the PSO procedure.

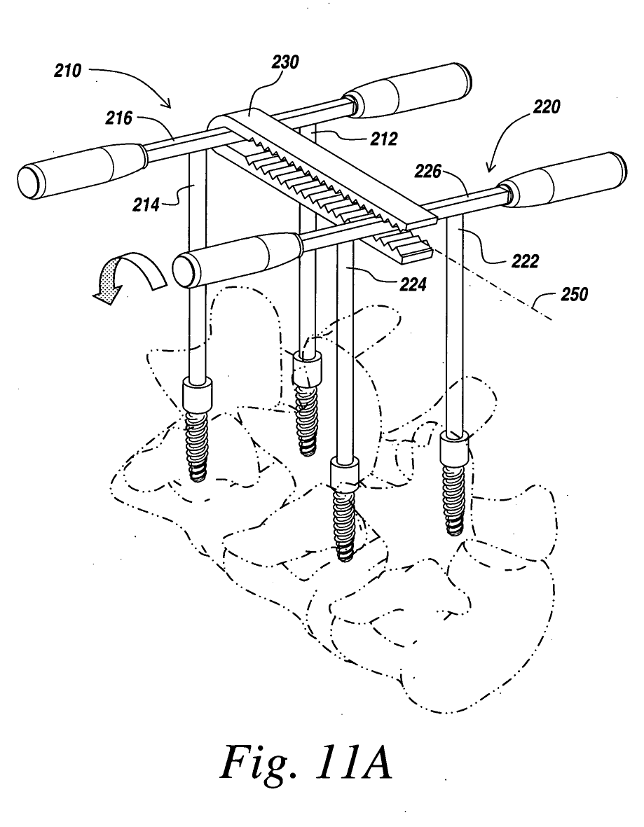

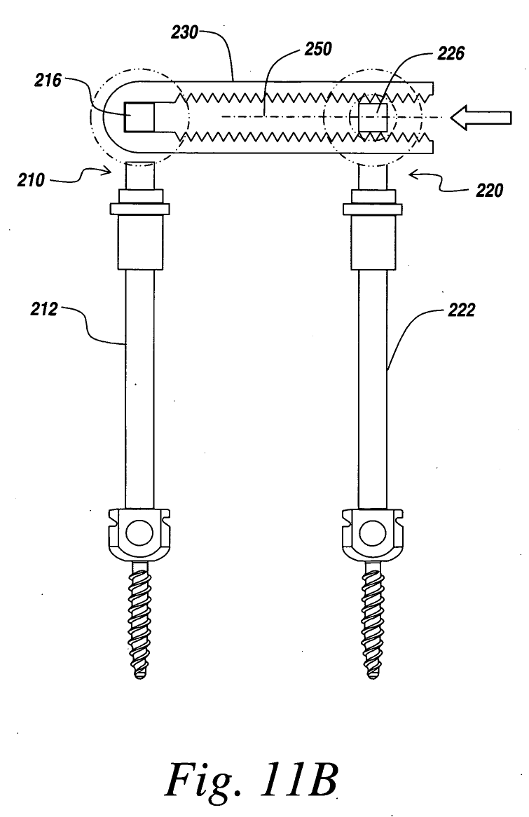

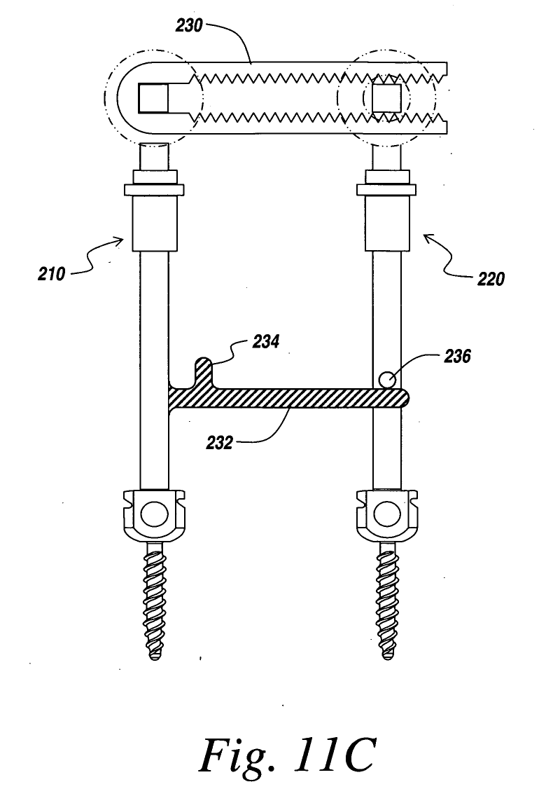

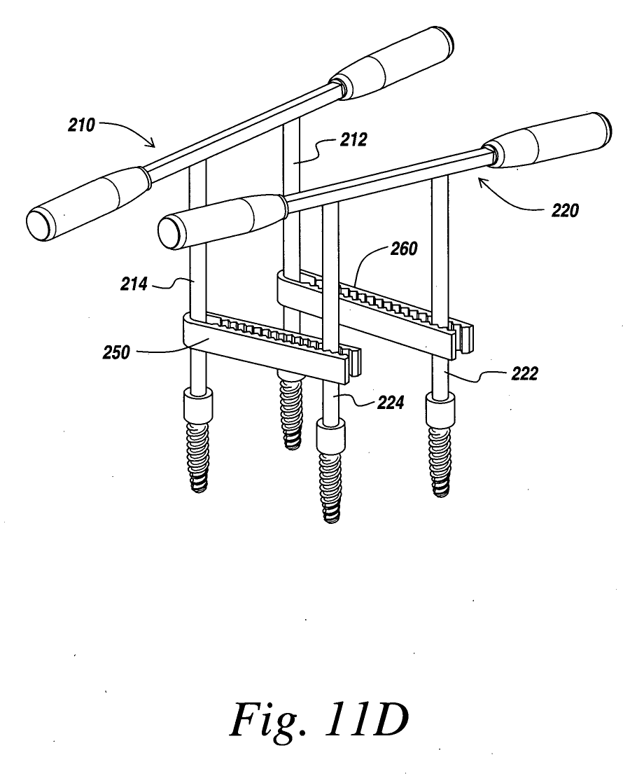

Two or more pelvic obliquity correction instruments can be used in conjunction, using a rack that links the pelvic obliquity correction instruments. FIG. 11A graphically depicts a perspective view of first and second pelvic obliquity correction instruments 210 and 220 linked by an exemplary rack 230. The first instrument 210 has a first engagement arm 212, a second engagement arm 214 and a handle assembly 216. The second instrument 220 has a first engagement arm 222, a second engagement arm 224 and a handle assembly 226. The engagement arms attach to the pelvic construct over screw heads, under screw heads, on transverse or longitudinal rods, or directly to bone anchors.

The rack 230 links the instruments 210 and 220, and allows motion of the instruments 210 and 220 in an axis 250 that lies along the rack 230. A first end of the rack 230 is connected to a portion of the handle assembly 216 of the first instrument 210, and a second end of the rack 230 is connected to a portion of the handle assembly 226 of the second instrument 220.

FIG. 11B graphically depicts a side view of the rack 230 which is connected to the first engagement arm 212 of the first instrument 210 and the first engagement arm 222 of the second instrument 220. Force can be applied to the rack 230 along the axis 250 to reduce the spacing between the first and second instruments 210 and 220. This effectively rotates the level of a pelvic construct attached to one of the instruments 210 and 220 relative to a level of a pelvic construct attached to the other instrument, and induces lordosis.

FIG. 11C graphical depicts a side view of a rack 230 which includes a guide 232 to control or limit translation of one of the instruments 210 and 220 relative to the other instrument. The guide 232 is disposed on an engagement arm of instrument 210 or instrument 220. A pin 236 is disposed on an engagement arm of the other instrument. The guide 232 has a protrusion 234 which stops relative movement of the instruments 210 and 220 when the pin 236 reaches and stops at the protrusion 234. The positions of the guide 232, the protrusion 234 and the pin 236 can be adjusted to permit a user to “dial in” the desired motion.

FIG. 11D graphically depicts a perspective view of first and second pelvic obliquity correction instruments 210 and 220 whose left sides are linked by a first rack 250 and whose right sides are linked by a second rack 260. The first rack 250 links together a first engagement arm 214 of the first instrument 210 and a first engagement arm 224 of the second instrument 220. The second rack 260 links together a second engagement arm 212 of the first instrument 210 and a second engagement arm 222 of the second instrument 220.

Although various components of exemplary spinal correction instruments are depicted in particular combinations, one of ordinary skill in the art will recognize that other combinations of the various components fall within the scope of the present invention. Although techniques for using exemplary instruments are described with respect to pelvic obliquity correction, one of ordinary skill in the art will appreciate that exemplary embodiments of the instrument described herein may also be used for other surgical techniques involving the spine and/or the pelvic construct.

One skilled in the art will appreciate further features and advantages of the invention based on the above-described embodiments. Accordingly, the invention is not to be limited by what has been particularly shown and described, except as indicated by the appended claims.

While the instruments and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

We claim:

1. A pelvic obliquity correction instrument, comprising:
a first engagement arm having an engagement end for engaging a first section of a spinal fixation system secured to a patient and having an adjustable coupling;
a second engagement arm having an engagement end for engaging a second section of the spinal fixation system and having a coupling portion; and
a handle assembly coupling the first engagement arm and the second engagement arm, the handle assembly comprising:
a first handle portion coupled with the first engagement arm;
a second handle portion coupled with the second engagement arm; and
a joint rotatably coupling the first handle portion and the second handle portion of the handle assembly;
wherein the first engagement arm adjustably couples with the handle assembly and is configured to adjust a distance between the first engagement arm and the second engagement arm.

2. The instrument of claim 1, wherein the first handle portion of the handle assembly adjustably couples with the adjustable coupling of the first engagement arm, and wherein the second handle portion of the handle assembly couples with the coupling portion of the second engagement arm.

3. The instrument of claim 1, wherein the joint is a polyaxial joint to permit rotation about more than one rotational axis.

4. The instrument of claim 1, further comprising a handle locking element to lock an orientation of the first handle portion with respect to the second handle portion.

5. The instrument of claim 1, wherein the adjustable coupling of the first engagement arm is slidably coupled with the handle assembly.

6. The instrument of claim 5, wherein the adjustable coupling between the first engagement arm and the handle assembly includes a frictional drag feature to resist sliding of the first engagement arm relative to the handle assembly.

7. The instrument of claim 1, wherein the second engagement arm includes an adjustable coupling disposed at the coupling portion of the second engagement arm.

8. The instrument of claim 1, wherein the engagement end of the first engagement arm is configured to engage a bone anchor.

9. The instrument of claim 1, wherein the engagement end of the first engagement arm is configured to engage a rod of the spinal fixation system.

10. The instrument of claim 1, wherein a slot for engaging a spinal fixation element is formed in the engagement end of the first engagement arm.

11. The instrument of claim 10, wherein the slot has an orientation that is substantially perpendicular to a longitudinal orientation of the first engagement arm.

12. The instrument of claim 10, wherein the slot has an orientation that is substantially parallel to a longitudinal orientation of the first engagement arm.

13. The instrument of claim 1, wherein the first engagement arm and the second engagement arm are curved with an engagement end separation distance between the engagement end of the first engagement arm and the engagement end of the second engagement arm that is smaller than a coupling end separation distance between the adjustable coupling of the first engagement arm and the coupling portion of the second engagement arm.

14. The instrument of claim 1, wherein the handle assembly further comprises:
a first grip and a second grip.

15. The instrument of claim 14, wherein the first grip and the second grip are separable from the handle assembly for replacement with a different first grip and a different second grip.

16. The instrument of claim 1, wherein the first engagement arm comprises a coupling piece and an engagement piece that is separable from the coupling piece.

17. The instrument of claim 16, wherein the coupling piece and the engagement piece are configured to separate from each other and rejoin with the engagement piece rotated about a longitudinal axis of the engagement piece for changing an orientation of the engagement end of the first engagement arm relative to the handle assembly.

18. A surgical instrument for positioning a first portion of a patient's spine relative to a second portion of the patient's spine, the surgical instrument comprising:
a first engagement arm for engaging a first portion of a spinal fixation system secured to a patient;
a second engagement arm for engaging a second portion of the spinal fixation system;
a handle assembly connecting the first engagement arm and the second engagement arm; and
a joint coupling the first engagement arm and a second engagement arm.

19. A method for correcting pelvic obliquity, comprising:
providing a pelvic obliquity correction instrument, the pelvic obliquity correction instrument comprising:
a first engagement arm having an engagement end for engaging a first section of a spinal fixation system and having an adjustable coupling disposed at a coupling end of the first engagement arm;
a second engagement arm having an engagement end for engaging a second section of the spinal fixation system and having a coupling end; and
a handle assembly having a first grip disposed at a first end and a second grip disposed at a second end and coupling the first engagement arm and the second engagement arm;
wherein the adjustable coupling of the first engagement arm adjustably couples with the handle assembly and is configured to adjust an engagement end separation distance between the engagement end of the first engagement arm and the engagement end of the second engagement arm;
adjusting the engagement end separation distance to approximately equal a distance between the first section of the spinal fixation system and the second section of the spinal fixation system previously attached to a patient;
engaging the first section of the spinal fixation system with the engagement end of the first engagement arm;
engaging the second section of the spinal fixation system with the engagement end of the second engagement arm;
exerting forces on the first grip and the second grip to reposition a patient's pelvic bone with respect to a superior portion of the patient's spine;
fixing a position of the pelvic bone of the patient with respect to the superior portion of the spine of the patient; and
disengaging the pelvic obliquity correction instrument from the spinal fixation system.

20. A method for controlling a patient's spine during pedicle subtraction osteotomy, comprising:
providing a pelvic obliquity correction instrument, the pelvic obliquity correction instrument comprising:
a first engagement arm having a loading slot engaging a first section of a spinal fixation system and having an adjustable coupling;
a second engagement arm having a loading slow for engaging a second section of the spinal fixation system and having a coupling portion; and
a handle assembly having a first grip disposed at a first end and a second grip disposed at a second end and coupling the adjustable coupling of the first engagement arm and the coupling portion of the second engagement arm;
engaging the first section of the spinal fixation system with the engagement end of the first engagement arm;
engaging the second section of the spinal fixation system with the engagement end of the second engagement arm; and
inducing lordosis on the patient's spine by using the first and second grips to rotate the handle assembly or by pushing the first and second grips to move the handle assembly along an up/down axis.
